(19) United States
(12) Patent Application Publication
(10) Pub. No.: US 2007/0129736 A1
(43) Pub. Date: Jun. 7, 2007

(54) Adhesive textile implant for parietal repair

(76) Inventor: Gilles Solecki, Lyz lez Lannoy (FR)

Correspondence Address: LADAS & PARRY LLP, 224 SOUTH MICHIGAN AVENUE, SUITE 1600, CHICAGO, IL, 60604, US

(21) Appl. No.: 10/582,751

(22) PCT Filed: Dec. 14, 2004
(86) PCT No.: PCT/FR04/03218

§ 371 Date: Nov. 2, 2006

(30) Foreign Application Priority Data
Dec. 14, 2003 (FR) 0314695

(51) Int. Cl. A61F 2/02

(52) U.S. Cl. 606/151; 623/23.76

(57) ABSTRACT

An adhesive textile implant for parietal repair, which is used in the field of reconstructive surgery. The implant has a biocompatible textile structure including a coating or impregnation of a water-soluble biocompatible polymer composition having adhesive properties that remain when an implant is placed in the tissues of the organism. The implant is suitable for use as a hernia repair implant, neurological patch, etc.

This invention corresponds to the technique of surgical implants implantable as adhesive textiles intended for parietal repair of a human organism. Applications fall under not only the field of hernia treatment and abdominal ruptures, but also the treatment of urinary incontinence and vaginal and rectal prolapse, plastic surgery of the rachidian and cerebral dura mater by means of patches, plastic surgery of the pericardium and repairs of soft tissues in orthopedics.

Until now, surgical implants for parietal repairs are secured by stapling, sutures, or taping. Securing is done by a surgeon with the help of a separate medical device: stapler, thread, vaporizer.

It is possible to not secure the implant, but the risk of the parietal implant moving then increases.

The staples, as described in the international request for patent WO 03/034925, whereas the surgical sutures offer a better way of securing the implant on the tissues, that they be biocompatible, resorbing or not.

However, stapling remains traumatic, a nervous termination can be pinched and you may sometimes suffer from post-operative pain. Secondary adhesions can appear on the staples, especially for rupture plates placed intra-peritoneal.

Moreover, suturing has the inconvenience of being a long operation.

Surgical adhesives to tape an implant to human tissues, such as fibrin-based and cyanoacrylate-based glues are already well known.

Fibrin-based adhesives, completely biodegradable, are only poor adhesives in comparison to cyanoacrylate glues. Fibrin-based glues are applied by the surgeon on the implant and they require an extensive and restrictive initial preparation for the operating nurse.

Cyanoacrylate-based glues have a very strong adhesion, but necrotize living tissue or burn them by exothermic reaction. The speed at which cyanoacrylate hardens is an obstacle when using it, as the repositioning of the plate after contact is no longer possible. Biocompatibility is not proven, as the exothermic hardening reaction releases certain toxic molecules.

The main disadvantage of these glues remains the problem with the dose and application during use, as the adhesive needs to be laid down on the plate, under operating conditions in the operating room.

The invention consists of a textile surgical implant for parietal repairs whose adhesive properties vary with the product's environment. The implant is offered in a variety of forms, not only limited to these examples, as a plate for hernia repairs, a patch for dura mater plastic surgery, a gynecological implant impregnated with biocompatible adhesive.

In the packaging, the biocompatible adhesive, which covers the implant, is inactive. Once inside the organism, the covering adhesive properties are activated when the surgeon places it on internal tissues. Indeed, the simultaneous compressive action of force that the surgeon exerts on the implant combined with humidity of the tissues activates the adhesive properties of the polymeric bio-adhesive composition coated on the textile or impregnated in the textile that make up the implant.

The bio-adhesive polymeric composition used is a water-soluble polymeric composition has the aptitude to make the implant adhere in a way that it can be repositioned on tissues of the human organism only under the combined action of water molecules and compressive force.

This bio-adhesive is advantageously an adhesive sensitive to pressure or P.S.A. adhesive (Pressure Sensitive Adhesive).

Preferably, the bio-adhesive used includes polyvinylpyrrolidone (P.V.P.), a polymer having these particular adhesive properties.

In the internal tissue environment of the human organism, characterized by the presence of water and compressive forces exerted by the internal organs or muscles, the adhesive properties of such adhesives have a persistent and effective action while permitting the repositioning of the implant by the surgeon grasping the latter.

The adhesive properties can eventually be adjusted by adding polyethylene glycol (P.E.G.) in selected proportions.

Indeed, it was discovered that P.E.G. causes a drop in the solution's dynamic viscosity, plays the role of plasticizer and allows the formation of a “scalable” structure between the P.V.P. and P.E.G. chains thanks to their water-absorbent sites, which makes the implant flexible.

Also, polyvinylpyrrolidone, with or without polyethylene glycol, that belongs to pressure-sensitive adhesives can also be used.

As an example of proportion, given P.V.P. with a molecular weight of 106 grams per mole and P.E.G. with a molecular weight of 400 grams per mole, the ideal proportion making it possible to obtain the most adherent mixture is 64 percent of P.V.P for 36 percent of P.E.G. (mass proportions), at a relative humidity rate of 50-65%. The viscosity of the mixture can be controlled thanks to the quantity of water added: P.V.P. is polar and highly water-soluble.

Beyond 36%, the more you increase P.E.G. in proportion, the more the adherence is limited. However, to reduce the cost of a bio-adhesive polymeric composition, which includes P.V.P., and avoid too great of inflexibility of this one, the quantity of P.E.G. should not be too low. Viscosity tests, measured by shear stress on the Mettler viscometer, indicate that the P.V.P. and P.E.G. aqueous solution is shear viscous flowing and offers a thixotropic quality.

The solution obtained, in an ideal world, is sufficiently flowing to be applied, but also sufficiently viscous not to gush out, because its viscosity remains constant.

The taping of the implant, which contains the abovementioned adhesives, is created by the appearance of Van Der Waals types of chemical bonds or by hydrogen bonds, and not by covalent bonds. The covalent bonds are strong and do not allow the surgeon to easily reposition the implant, even if he wanted to, whereas the weak bonds generated by these adhesives are sufficient to maintain in place the implant on tissues undergoing the constraints, while allowing the repositioning of the implant.

The impregnation of the textile surgical implants in an aqueous polymer solution or bio-adhesive polymers thus described is done in clean room.

The textile structure of the implant is, for example, made up of knitted, woven or non-woven polypropylene or polyester material.

In order to favor the cellular recolonization making it possible for tissues of the organism to integrate and rebuild themselves through the textile structure of the implant, this one offers a porous structure.

The invention lets the surgeon or his assistants avoid the delicate operative phase of coating and applying the proper dose of glue on the implant, in an operating room, just before putting it in place, since the polymeric composition is already impregnated on the implant in a non-active state in an ambient environment.

The implant is already ready to be inserted and taped in the patient's organism and will develop its advantageous properties when it is put in place on the tissues.

The invention has the advantage of being an atraumatic bio-adhesive implant that is quickly secured and can easily be repositioned when it is put in place by the surgeon.

In a specific method of realization, the bio-adhesive is composed of polyvinylpyrrolidone.

The polyvinylpyrrolidone, which makes up the bio-adhesive, avoids the necrosis or burning of the tissues. Moreover, the breakdown of the bio-adhesive agent leaves room for fibrosis in a few weeks.

The surgical implant includes a biocompatible textile and at least a biocompatible polymeric composition, which is water-soluble and has the aptitude to make itself adhere to the implant on the tissues of the human organism, only under the combined action of water molecules and compressive force, so it can be repositioned.

The polymeric composition quite advantageously includes a polymeric adhesive from the adhesive family sensitive to pressure.

The biocompatible polymeric composition is impregnated on at least one part of the implant or coated onto at least one of the surfaces of the implant.

The self-adhesive biocompatible polymeric composition can be mixed with active pharmaceutical agents (for example, antibiotics, anti-cancerous agents, auto coagulants).

The polymeric composition can include polyvinylpyrrolidone (P.V.P.), and, can include, as an alternative, a mixture of polyvinylpyrrolidone (P.V.P.) and polyethylene glycol (P.E.G.), which can advantageously replace P.V.P. used by itself.

In a design variant, the polymeric composition can include carboxymethylcellulose (C.M.C.).

It is possible to use, as a polymeric composition, a mixture of polymers made up of carboxymethylcellulose (C.M.C.) and polyethylene glycol (P.E.G.).

The self-adhesive biocompatible polymeric composition can also include a copolymer made up of monomers belonging to the acrylate and monomer family selected to give water solubility to the self-adhesive biocompatible polymer.

The acrylate monomer can be selected from the group category of Octyl acrylate, 2-Ethylhexyl acrylate, Isooctyl acrylate, Isononyl acrylate, Hexyl acrylate, Butyl acrylate, and the monomer selected to give water solubility to the self-adhesive polymeric composition is selected from the group category of β-acryloyloxy propionic acid, acrylic acid, vinylphosphonic acid, methacrylic acid.

The self-adhesive biocompatible polymeric composition can also include a copolymer made up of monomers belonging to the category of acrylates, monomers selected to give water solubility to the self-adhesive polymer as well as to Hydroxyalkyl(meth)acrylate monomers.

The acrylate monomer can be selected from the group category: Octyl acrylate, 2-Ethylhexyl acrylate, Isooctyl acrylate, Isononyl acrylate, Hexyl acrylate, Butyl acrylate; the monomer selected to give water solubility to the self-adhesive polymeric composition can be selected from the group category of: β-acryloyloxy propionic acid, acrylic acid, vinylphosphonic acid, methacrylic acid; the hydroxyalkyl(meth)acrylate monomer can be selected from the group category of: 2-hydroxyethyl acrylate, 2-hydroxypropyl acrylate, the 2-hydroxyethyl methacrylate, 2-hydroxypropyl methacrylate.

Among the different manufacturing methods, the first one possible is to make the invention by the technique of impregnation: the biocompatible polymeric composition is impregnated in the core of the textile surgical implant, by soaking the structure in an aqueous solution in at least one biocompatible polymer for a few seconds. The implant is then suspended out to dry in a kiln for at least 24 hours, at a temperature of 50° Celsius [122° F.].

In a second possible method of realization, the invention is made by coating an aqueous solution of at least one polymer.

In a nonrestrictive example of realization, the aqueous solution of at least one polymer contains for 100 grams of demineralized water 165 grams of P.V.P. grade K30 (molecular weight between 44 and 58 kilograms per mole) and 19 grams of P.E.G. with a molecular weight of 400 grams per mole.

The drying of the implant thus obtained is done on a flat surface, drying in a kiln at 50° Celsius [122° F.] for at least 24 hours. Resulting in a film of at least one polymer deposited on the surface of the fibers of the implant.

It goes without saying that many variations can be applied to it, in particular by substituting equivalent technical measures, without working outside the framework the invention.

1. Surgical implant, including a textile and a biocompatible polymeric composition, wherein the polymeric composition is water-soluble and has the aptitude to make the implant adhere, in a way that it can be repositioned onto tissues of the human organism only under the combined action of water molecules and compressive force.

2. Implant according to claim 1 wherein the biocompatible polymeric composition includes at least one adhesive pertaining to the group of adhesives sensitive to pressure.

3. Implant according to claim 1, wherein the biocompatible polymeric composition is impregnated on at least one part of the implant.

4. Implant according to claim 1, wherein the adhesive biocompatible polymeric composition is coated on at least one of the surfaces of the implant.

5. Implant according to claim 1, wherein the self-adhesive biocompatible polymeric composition is mixed with active pharmaceutical agents.

6. Implant according to claim 1, wherein the polymeric composition includes polyvinylpyrrolidone (P.V.P.).

7. Implant according to claim 1, wherein the polymeric composition includes a mixture of polyvinylpyrrolidone (P.V.P.) and polyethylene glycol (P.E.G.).

8. Implant according to claim 1, wherein the polymeric composition includes carboxymethylcellulose (C.M.C.).

9. Implant according to claim 8, wherein the polymeric composition includes carboxymethylcellulose (C.M.C.) mixed with polyethylene glycol (P.E.G.).

10. Implant according to claim 1, wherein the self-adhesive biocompatible polymeric composition is a copolymer including monomers belonging to the acrylate and monomer family selected to give water solubility to self-adhesive biocompatible polymer.

11. Implant according to claim 10, wherein the acrylate monomer is selected from the group consisting of: Octyl acrylate, 2-Ethylhexyl acrylate, Isooctyl acrylate, Isononyl acrylate, Hexyl acrylate, and Butyl acrylate, and wherein the monomer selected to give water solubility to the self-adhesive polymer is selected from the group consisting of: β-acryloyloxy propionic acid, acrylic acid, vinylphosphonic acid, and methacrylic acid.

12. Implant according to claim 10, wherein the self-adhesive polymeric composition includes moreover Hydroxyalkyl(meth)acrylate monomers.

13. Implant according to claim 12, wherein the hydroxyalkyl(meth)acrylate monomer is selected from the group consisting of: 2-hydroxyethyl acrylate, 2-hydroxypropyl. acrylate, 2-hydroxyethyl methacrylate, and 2-hydroxypropyl methacrylate.
